(19) United States
(12) Patent Application Publication
(10) Pub. No.: US 2012/0292563 A1
(43) Pub. Date: Nov. 22, 2012

(54) PROCESS FOR PRODUCING CARBON NANOTUBES CONTAINING HYDROXYALKYL ESTER GROUPS AND MATERIALS AND DISPERSIONS CONTAINING SAID CARBON NANOTUBES

(75) Inventors: Stephanie Vogel, Langenfeld (DE); Hartmut Nefzger, Pulheim (DE); Jörg Hofmann, Krefeld (DE)

(73) Assignee: BAYER INTELLECTUAL PROPERTY GMBH, Monheim (DE)

(21) Appl. No.: 13/522,438

(22) PCT Filed: Jan. 10, 2011
(86) PCT No.: PCT/EP2011/050231

§ 371 Date: Jul. 16, 2012

(30) Foreign Application Priority Data
Jan. 16, 2010 (DE) 102010004806.2

(51) Int. Cl. C07C 67/26; C08G 18/10; C09K 3/00; C04B 35/80; B82Y 30/00; B82Y 40/00

(52) U.S. Cl. 252/182.24; 560/8; 501/1; 106/287.24; 528/59; 977/847; 977/746; 977/779

(57) ABSTRACT

The invention relates to a process for the preparation of carbon nanotubes having hydroxyalkyl ester groups by reacting carbon nanotubes which have carboxylic acid groups with one or more epoxides, and to dispersions and materials which contain the carbon nanotubes functionalized thus.

The present invention relates to a process for producing carbon nanotubes containing hydroxyalkyl ester groups, a process for producing dispersions containing these carbon nanotubes, particularly preferably dispersions in polyols and/or polyisocyanates, wherein optionally some of the carbon nanotubes can be covalently bonded to the polyisocyanate dispersing agent, a process for producing materials containing these carbon nanotubes, particularly preferably polyurethane polymers, and materials containing carbon nanotubes, wherein optionally some of the carbon nanotubes are covalently bonded to the material, in particular polyurethane polymer.

Carbon nanotubes (CNTs) are known for their exceptional properties. For instance, their strength is roughly 100 times that of steel, their thermal conductivity is around twice that of diamond, their thermal stability is up to 2800° C. under vacuum and their electrical conductivity can be many times that of copper. At a molecular level, however, these structure-related characteristics are only accessible if the carbon nanotubes can be homogeneously distributed and the greatest possible contact established between the tubes and the medium, in other words by making them compatible with the medium and hence stably dispersible.

A chemical functionalisation of carbon nanotubes or carbon fibres can improve their dispersibility among other things. A review article by N. Tsubokawa (Polymer Journal 2005, 37, 637-655) lists a large number of possibilities for such a functionalisation. In addition to complex chemical reactions such as for example ligand exchange reactions at 1,1′-dicarboxyferrocene, living radical polymerisation with styrene and reaction with azides, mention was also made of the oxidation of carbon nanotubes with HNO₃ and corresponding modifications based thereon, as is known in this field. Tsubokawa reports on three possibilities for subsequent chemistry following the oxidative introduction of carboxyl groups.

The first variant is the reaction of the carboxyl groups of the carbon nanotubes with coupling reagents such as dicyclohexyl carbodiimide known from peptide chemistry, with subsequent reaction with a nucleophile. However, this method requires the use of an expensive coupling reagent and, depending on the coupling reagent used, produces a poorly-soluble urea as a by-product.

The second variant is the activation of the acid groups with thionyl chloride, followed by a further reaction with a nucleophile. The disadvantage here is the release of SO₂ and HCl caused by the use of thionyl chloride. Furthermore, HCl is released again in the subsequent reaction of this activated acid function with the nucleophile. This by-product would have a disruptive effect on a subsequent use of carbon nanotubes functionalised in this way in dispersions and would influence chemical reactions.

Finally a third variant is described in this review article by reference to carbon fibres obtained from the gas phase (vapour-grown carbon fibres, VGCF). Here an anionic copolymerisation of epoxides and acid anhydrides takes place by means of alternating ring opening. This has already been described separately by the same author in Polymer Journal 2004, 36, 316-322. The synthesis sequence is initiated by the deprotonation of the carboxyl group with KOH. This means that polymerisation has to be performed in the presence of crown ethers, which makes this chemistry very expensive and leads to problems with waste disposal. In the specific example the carboxylate group of the carbon fibre is reacted with styrene oxide and phthalic anhydride. A table indicates that the reaction of the carboxylate group with styrene oxide alone fails to bring about a conversion.

The functionalisation of carboxyl groups in polyester polyol synthesis is disclosed in DE 36 13 875 A1. Polycarboxylic acids and/or anhydrides thereof and polyhydric alcohols are condensed to produce polyester polyols with an acid value of less than 1, a hydroxyl value of approximately 20 to approximately 400 and a functionality of conveniently 2 to 3. This takes place advantageously in the absence of conventional esterification catalysts at temperatures of 150° C. to 250° C. and optionally under reduced pressure. Polycondensation is continued until an acid value of 20 to 5 is achieved, and the polycondensates obtained are then alkoxylated with 1 to 5 mol per carboxyl group of alkylene oxide, for example 1,2-propylene oxide and/or preferably ethylene oxide, in the presence of a tertiary amine The tertiary amine is selected from the group comprising N-methylimidazole, diazabicyclo-[2,2,2]octane, diazabicyclo [5,4,0]undec-7-ene and pentamethylene diethylenetriamine. The catalyst is conveniently used in an amount of 0.001 to 1.0 wt. %, relative to the weight of polycondensate. Alkoxylation is advantageously performed at temperatures of 100° C. to 170° C. and under a pressure of 1 to 10 bar.

The object was to provide an improved process for producing functionalised carbon nanotubes in which, building on carboxyl groups located at the surface, hydroxyalkyl ester groups are present in which there is a distance of at least two carbon atoms between the ester function and the free OH group.

The invention provides a process for producing carbon nanotubes containing hydroxyalkyl ester groups comprising the following steps:
(a) provision of carbon nanotubes having covalently bonded carboxyl groups at their surface; and
(b) reaction of the carbon nanotubes from step (a) with one or more epoxides

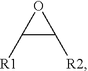

wherein R1 and R2 independently of each other are hydrogen, an alkyl radical or aryl radical and the epoxides are in gaseous form during the reaction.

The carbon nanotubes provided in step (a) are preferably in solid form.

In connection with this invention the term “alkyl” encompasses substituents from the group comprising n-alkyl, branched alkyl and cycloalkyl. In connection with this invention the term “aryl” encompasses substituents from the group comprising mononuclear carboaryl and heteroaryl and polynuclear carboaryl and heteroaryl.

The advantage of the process according to the invention over the prior art is the cost-effective synthesis route via the gas phase. In the process according to the invention activation as acid chloride is not necessary, so this step as performed in the prior art is dispensed with. The starting material and end product are solids which can be incorporated universally into any medium with no further processing. The reaction of gaseous reactants with the oxidised carbon nanotubes used in solid form in the process according to the invention avoids the need for very complex processing steps. In the prior art, by contrast, the functionalised carbon nanotubes have to be isolated from a liquid reaction medium or solvent on completion of the reaction by very complex means. The large surface area of carbon nanotubes makes it more difficult to remove the excess medium or solvent completely, meaning that traces of this adsorbed material remain adhering to them. The reaction of the oxidised carbon nanotubes via the gas phase is therefore particularly advantageous, as impurities of this type are avoided in this case and the solid product can be used universally in any medium. There is a further advantage in comparison to production via acid chlorides, in that the process according to the invention is an addition reaction, which requires very small amounts of a catalyst, which is likewise in gaseous form during the reaction. In the case of esterification via acid chloride (prior art), stoichiometric amounts of hydrogen chloride are released which have to be removed in order to prevent subsequent reactions. Surprisingly the process according to the invention can also be performed without catalysis.

The carbon nanotubes functionalised according to the invention can be dispersed directly into a hydroxy-group-reactive or non-reactive medium. Surprisingly it has been found that more homogeneous dispersions having greater long-term stability are obtained with the carbon nanotubes functionalised by the process according to the invention as compared with those with non-functionalised carbon nanotubes. It has also been found that the filler-induced rise in viscosity in the dispersion with the carbon nanotubes functionalised by the process according to the invention is much smaller than in that with the non-functionalised carbon nanotubes. Polyurethanes filled with the carbon nanotubes functionalised according to the invention surprisingly have greatly improved mechanical properties, even with low filler concentrations. Moreover, despite the chemical functionalisation of the carbon nanotubes, the resistivity of polyurethanes filled therewith is surprisingly demonstrably reduced.

The carbon nanotubes having carboxyl groups covalently bonded to the surface which are used as the starting material can be obtained from non-functionalised carbon nanotubes by means of oxidative processes, such as the HNO₃ process, for example. The content of carboxyl groups at the surface can be determined by conductometric titration and is stated in mmol of carboxyl groups per gram of carbon nanotubes. The content can preferably be ≧0.01 mmol/g to ≦50 mmol/g, particularly preferably≧0.1 mmol/g to ≦10 mmol/g.

In step (b) of the process according to the invention the gaseous epoxide can advantageously be used in a large excess relative to the carboxyl groups on the surface of the carbon nanotubes. The reaction pressure (absolute) can preferably be between ≧1 bar and ≦10 bar, particularly preferably between ≧1 bar and ≦6 bar. The reaction temperature can preferably be between ≧50° C. and ≦200° C., particularly preferably between ≧80° C. and ≦150° C. The reaction can preferably be performed under protective gas (e.g. nitrogen, argon, helium).

In a preferred embodiment of the process according to the invention the reaction of the carbon nanotubes in step (b) is performed in the presence of a gaseous tertiary amine as catalyst. Tertiary amines are preferred. Examples thereof are diazabicycloalkanes, such as 1,4-diazabicyclo[2.2.2]octane, diazabicycloalkenes, such as 1, 8-diazabicyclo[5.4.0]undec-7-ene, triamines, such as pentamethyldiethylenetriamine, bis(2-dialkylaminoalkyl)ethers, such as bis(2-dimethylaminoethyl)ether, and trialkylamines, such as triethylamine, trimethylamine, N,N-diethylmethylamine or N,N-dimethylethylamine. The catalyst is particularly preferably N,N-dimethylethylamine.

In a preferred embodiment R1 and R2 in the epoxide in step (b), which is in gaseous form during the reaction, independently of each other denote hydrogen, methyl, ethyl, n-propyl, isopropyl, n-butyl, sec-butyl, isobutyl, tert-butyl or phenyl or together denote —(CH₂)₄13 . The epoxide in step (b) is particularly preferably a terminal alkylene oxide. Examples of such alkylene oxides are ethylene oxide, propylene oxide, 1,2-butylene oxide and 2,3-butylene oxide. Ethylene oxide and propylene oxide are particularly preferred.

In a further preferred embodiment the carbon nanotubes in step (a) are selected from the group encompassing single-walled carbon nanotubes, multi-walled carbon nanotubes, multi-walled carbon nanotubes of the cylinder type, scroll type, multi-scroll type and/or with an onion-like structure. Carbon nanotubes having a ratio of length to external diameter of ≧5, preferably ≧100, are particularly preferred.

Carbon nanotube structures consisting of multiple graphite layers which are stacked together and rolled up can likewise be used. These are referred to as multi-scroll carbon nanotubes. (NB: These are already mentioned in the previous paragraph.) These carbon nanotubes are described in DE 10 2007 044031 A1.

The carbon nanotubes containing hydroxyalkyl ester groups produced according to the invention preferably have a diameter of ≧3 nm to ≦100 nm, particularly preferably ≧4 nm to ≦80 nm, most particularly preferably ≧5 nm to ≦60 nm. The diameter relates here to the average diameter of the carbon nanotubes. There is no limit to the length of the carbon nanotubes. It can preferably be in a range from ≧1 μm to ≦100 μm and particularly preferably from ≧10 μm to ≦30 μm.

The carbon nanotubes containing free OH groups produced by the process according to the invention can be reacted further chemically to the hydroxy groups if required. The content of free OH groups, expressed in mmol of OH groups per gram of carbon nanotubes, is preferably ≧0.01 mmol/g to ≦50 mmol/g, preferably ≧0.1 mmol/g to ≦10 mmol/g.

Methods for the homogeneous distribution and mixing of the carbon nanotubes containing hydroxyalkyl ester groups produced according to the invention in a solid matrix are generally known. These can be grinding processes under very high pressure adapted to the individual conditions, for example. Suitable solid matrices are for example inorganic polymers, such as ceramics, or polymeric inorganic oxides, such as aluminium oxides for example. Silicon carbides, silicon nitrides and boron nitrides and mixtures thereof are likewise suitable. Furthermore, metals, such as for example aluminium, magnesium, lead, copper, tungsten, titanium, niobium, hafnium, vanadium, silver and mixtures thereof, are suitable as the solid matrix for the homogeneous distribution of the carbon nanotubes produced according to the invention.

The present invention also provides the use of the carbon nanotubes containing hydroxyalkyl ester groups produced according to the invention in various liquid or meltable media for the production of dispersions. The carbon nanotubes functionalised according to the invention can be covalently bonded in the medium/matrix via the free hydroxy groups. However, it is just as possible for the carbon nanotubes according to the invention to be present in the medium/matrix without covalent bonding. The usual dispersing methods for incorporation are generally known. Incorporation can take place for example by the application of high shear forces using a rotor-stator system, jet disperser, extruder or calender, by means of grinding processes using a ball and/or bead mill, or by the application of cavitation forces via ultrasound. Liquid or meltable media that can be used are for example raw materials for organic polymers or thermoplastic polymers or meltable thermoplastic polymers. Polyamides, polycarbonates, polyesters, polyethers, polyimides, polyphenylenes, polysulfones, polyurethanes, epoxy resins, rubbers or mixtures thereof, for example, are preferred. Dispersions in polyurethane (PU) raw materials (as the medium) are particularly preferred. Suitable PU raw materials are both polyols and polyisocyanates. The concentration of carbon nanotubes in a dispersion is preferably in a range from ≧0.01 wt. % to ≦10 wt. %, particularly preferably from ≧0.1 wt. % to ≦5 wt. %. The dispersions can be obtained using a stirrer with a rotor-stator system at high speeds, such as for example between ≧2000 rpm and ≦30,000 rpm. The dispersion can additionally be exposed to ultrasound.

Suitable polyols are in principle the conventional polyols in polyurethane chemistry, such as for example polyether, polyacrylate, polycarbonate, polycaprolactone and polyester polyols. These polyols are described in “Ullmanns Enzyklopädie der technischen Chemie”, 4th Edition, Volume 19, p. 304-305, Verlag Chemie, Weinheim, or in “Polyurethane—Lacke, Kleb—and Dichtstoffe” by Ulrich Meier-Westhues, Vincentz Network, Hanover, 2007.

Suitable polyisocyanates are aromatic, araliphatic, aliphatic or cycloaliphatic polyisocyanates having an NCO functionality of ≧2.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(isocyanatocyclohexyl)methanes or mixtures thereof with any isomer content, 1,4-cyclohexylene diisocyanate, 1,4-phenylene diisocyanate, 2,4- and/or 2,6-toluylene diisocyanate (TDI), 1,5-naphthylene diisocyanate, 2,2′- and/or 2,4′- and/or 4,4′-diphenylmethane diisocyanate (MDI), 1,3- and/or 1,4-bis-(2-isocyanatoprop-2-yl)benzene (TMXDI), 1,3-bis-(isocyanatomethyl)benzene (XDI), and alkyl-2,6-diisocyanatohexanoates (lysine diisocyanates) having C₁ to C₈ alkyl groups.

In addition to the aforementioned polyisocyanates, modified diisocyanates having a uretdione, isocyanurate, urethane, allophanate, biuret, iminooxadiazinedione and/or oxadiazinetrione structure and non-modified polyisocyanate having more than 2

NCO groups per molecule, such as for example 4-isocyanatomethyl-1,8-octane diisocyanate (nonane triisocyanate) or triphenylmethane-4,4′,4″-triisocyanate, can be used as part or all of the polyisocyanate component.

It is also possible for NCO-terminated prepolymers consisting of the aforementioned polyisocyanates and polyols to be used as the polyisocyanate component.

In a further preferred process variant the carbon nanotubes produced according to the invention are particularly preferably dispersed in polyisocyanates, the dispersion is then heated, optionally using a catalyst, to temperatures of between 60° and 150° C., wherein at least some of the terminal free hydroxy groups of the carbon nanotubes react with the polyisocyanate, and then cooled. An additional dispersion can take place after the reaction if necessary.

The carbon nanotubes containing hydroxyalkyl ester groups produced according to the invention can particularly advantageously be used in the synthesis of polyurethane polymers and can thus be incorporated covalently into the polymer matrix.

Therefore the present invention also provides a process for producing polyurethane polymers containing carbon nanotubes functionalised according to the invention, comprising the following steps:
(a) provision of a first dispersion of carbon nanotubes functionalised according to the invention (i) in a polyol or (ii) in a polyisocyanate,
(b) reaction of this first dispersion (i) with a polyisocyanate or (ii) with a polyol and/or a second dispersion of carbon nanotubes functionalised according to the invention (i) in a polyisocyanate or (ii) in a polyol, optionally in the presence of the catalysts, chain extenders, crosslinking agents, auxiliary substances, such as for example stabilisers, flame retardants, antioxidants, and solid and/or liquid additives, such as for example pigments, carbon blacks, graphites, conductive carbon blacks, graphenes, non-functionalised carbon nanotubes, plasticisers commonly used in polyurethane chemistry.

The carbon nanotubes functionalised according to the invention can preferably be present in both components (polyol and polyisocyanate), wherein these two dispersions are then reacted with each other, optionally together with additional polyisocyanate and/or polyol.

The molar ratio of NCO groups in the polyisocyanate to NCO-reactive OH groups is preferably ≧0.90:1 to ≦4.50:1, particularly preferably ≧0.95:1 to ≦3.50:1, most particularly preferably ≧0.95:1 to ≦1.5:1.

The polyol in step (a) or (b) is preferably a polyether polyol and/or a polyester polyol. Preferred polyether polyols have hydroxyl values of ≧25 mg KOH/g to ≦550 mg KOH/g, particularly preferably ≧100 mg KOH/g to ≦520 mg KOH/g. Preferred polyester polyols have hydroxyl values of ≧100 mg KOH/g to ≦550 mg KOH/g, particularly preferably≧200 mg KOH/g to ≦500 mg KOH/g. The cited polyols preferably have molar masses in the range from ≧250 to ≦5000 g/mol, preferably ≧400 to ≦3500 g/mol, and a functionality between ≧1.8 and ≦6, preferably between ≧1.95 and ≦3.5.

The polyisocyanate in step (a) or (b) is preferably a polyisocyanate based on diphenylmethane diisocyanate (MDI). The fact that the polyisocyanate is based on MDI means that it is either monomeric, polynuclear and/or polymeric MDI. It can have an NCO content of ≧25 wt. % to ≦35 wt. %, for example. The NCO content can also be in a range from ≧29 wt. % to ≦31 wt. %.

A stiffening in comparison to a polyurethane not containing carbon nanotubes and also in comparison to one containing carbon nanotubes not functionalised according to the invention is surprisingly observed in the polyurethane polymer according to the invention.

The proportion of carbon nanotubes in the polyurethane polymer according to the invention is preferably ≧0.01 wt. % to ≦10 wt. %, particularly preferably ≧0.1 wt. % to ≦5 wt. %. Even such a small proportion of carbon nanotubes functionalised according to the invention in the polyurethane leads to a marked reinforcing of the polymer matrix.

The polyurethane polymer according to the invention can have an elasticity modulus of ≧1 N/mm² to ≦10,000 N/mm², for example. A polyurethane polymer having an elasticity modulus of ≧10 N/mm² to ≦5000 N/mm², preferably ≧100 N/mm² to ≦1000 N/mm², can be described as a polyurethane elastomer. The elasticity modulus is determined as a rise in the operating point of the stress-strain curve from the tensile test in accordance with DIN 53 504 with vanishing deformation between 0.025% and 0.05% elongation.

The present invention is illustrated in more detail by the examples below.

### EXAMPLES

The meanings of the materials and abbreviations used are as follows:
Desmophen® VP.PU 22HS51: Bifunctional polyether polyol with an OH value of 112 mg KOH/g (Bayer MaterialScience AG)
Desmodur® CD-S: Modified polyisocyanate based on 4,4′-diphenylmethane diisocyanate with an NCO content of 29.5% (Bayer MaterialScience AG)
DABCO 33-LV: Amine catalyst; 1,4-diazabicyclo[2.2.2]octane; 33% solution in propylene glycol (Air Products)
Niax A1: Amine catalyst; bis(2-dimethylaminoethyl)ether; 70% solution in dipropylene glycol (Momentive Performance Materials Inc.)
Dimethylethylamine N,N-Dimethylethylamine (Sigma Aldrich)
DBTL: Dibutyl tin dilaurate
DPG: Dipropylene glycol
CNT3: Baytubes® C150P, multi-walled carbon nanotubes (Bayer MaterialScience AG)
Oxidised CNTs: Oxidised carbon nanotubes (oxCNT) of the Baytubes® C150P type (Bayer MaterialScience AG) with carboxyl groups introduced at the surface by known methods (acid value =12.9 mg KOH/g)

The mechanical properties of the PU elastomers obtained in the examples were determined in accordance with the following standards:
Hardness [Shore A]: DIN 53 505
Tensile strength [MPa]: DIN 53 504
Maximum elongation [%]: DIN 53 504
Elasticity modulus [N/mm²]: As the rise in the operating point of the stress-strain curve from the tensile test in accordance with DIN 53 504 with vanishing deformation between 0.025% and 0.05% elongation.

The viscosities of the dispersions that were produced were determined using a Physica MCR 51 viscometer from Anton Paar and the corresponding electrical conductivities using a model 703 conductometer from Knick with a 4-pole measuring cell. The specified resistivities were calculated in accordance with ASTM D 257.

### General Notes on the Examples:

The Baytubes® C150P (multi-walled carbon nanotubes) oxidised by means of the HNO₃ process had a carboxyl group concentration of 0.23 mmol/g (conductometric titration).

Alkoxylation of the oxidised Baytubes took place by reaction with gaseous epoxides in a steel pressure reactor with amine catalysis or without catalysis over a longer period and with a higher excess of epoxide.

The alkoxylated carbon nanotubes obtained in this way were introduced into the polyol in the desired concentration immediately before reaction with the isocyanate and sheared with a rotor-stator system (T 18 basic ULTRA-TURRAX®, IKA Werke GmbH & Co. KG, Staufen, Germany) and treated with ultrasound (Probe Sonicator HD 3200, BANDELIN electronic GmbH & Co. KG, Berlin, Germany).

The dispersion thus obtained was degassed briefly under reduced pressure both before and after addition of the catalyst. The isocyanate was stirred briefly with the dispersion. This reaction mixture was poured into a hinged metal mould and annealed at 70° C.

The polyurethane elastomers containing carbon nanotubes obtained in this way were tested for their hardness and tensile strength and for their electronic properties.

### Example 1

(Alkoxylation, CNT1)→with Catalyst

75 g of the oxidised CNTs (oxCNT, acid value =12.9 mg KOH/g) were placed in a 1-litre steel pressure reactor under protective gas (nitrogen) and then heated to 125° C. whilst stirring (1200 rpm). 5.0 g of amine catalyst (dimethylethylamine) were added at this temperature and the mixture was stirred for 1 h at 125° C. Then the reactor was first depressurised to normal pressure and then pressurised five times with nitrogen to 5.0 bar (absolute) each time, returning the reactor to normal pressure again after each pressure build-up. Next the pressure was increased initially to 4.0 bar (absolute) nitrogen at 125° C. whilst stirring and then 2.5 g of ethylene oxide were added, causing the reactor pressure to rise to 4.3 bar (absolute). Once the pressure had dropped to 4.0 bar (absolute) again (after 5 min), a further 2.5 g of ethylene oxide were added, causing the reactor pressure to rise to 4.3 bar (absolute). Once the pressure had dropped to 4.1 bar (absolute) (after 7 min), the mixture was stirred for a further 60 min at 125° C. Then the mixture was cooled to room temperature, the reactor was depressurised to normal pressure and then pressurised five times with nitrogen to 5.0 bar (absolute) each time and returned to normal pressure again. The product was removed from the reactor and then treated for 2 hours under high vacuum at 150° C.

### Example 2

(Alkoxylation, CNT2)→without Catalyst

75 g of the oxidised CNTs (oxCNT, acid value =12.9 mg KOH/g) were placed in a 1-litre steel pressure reactor under protective gas (nitrogen) and then heated to 125° C. whilst stirring (1200 rpm). At this temperature the pressure was increased initially to 4.0 bar (absolute) nitrogen and then 2.5 g of ethylene oxide were added, causing the reactor pressure to rise to 4.3 bar (absolute). Once the pressure had dropped to 4.0 bar (absolute) again (after 4 min), a further 2.5 g of ethylene oxide were added three times, causing the reactor pressure to rise to 4.3 bar (absolute) each time and then drop to 4.1 bar. After stirring for a further 60 min at 125° C. the mixture was cooled to room temperature, the reactor was depressurised to normal pressure and then pressurised five times with nitrogen to 5.0 bar (absolute) each time and returned to normal pressure again. The product was removed from the reactor and then treated for 2 hours under high vacuum at 150° C.
Analytical values: Acid value =4.25 mg KOH/g

### Example 3

### (Dispersion, 1 Wt. % CNT1, Dispersion 1A)

99 g of Desmophen® VP.PU 22HS51 were added to 1.0 g of CNT1 in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 80 kJ. The dispersion 1A obtained in this way was used immediately for the further reaction with an isocyanate.

### Example 4

(Dispersion, 3 Wt. % CNT1, Dispersion 1B) 97 g of Desmophen° VP.PU 22HS51 were added to 3 g of CNT1 in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 240 kJ. The dispersion 1B obtained in this way was used immediately for the further reaction with an isocyanate.

### Example 5

### (Dispersion, 1 Wt. % CNT2, Dispersion 2A)

99 g of Desmophen° VP.PU 22HS51 were added to 1.0 g of CNT2 in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 80 kJ. The dispersion 2A obtained in this way was used immediately for the further reaction with an isocyanate.

### Example 6

### (Dispersion, 3 Wt. % CNT2, Dispersion 2B)

97 g of Desmophen® VP.PU 22HS51 were added to 3.0 g of CNT2 in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 240 kJ. The dispersion 2B obtained in this way was used immediately for the further reaction with an isocyanate.

### Example 7

### (Dispersion, 1 Wt. % CNT3, Dispersion 3A)

99 g of Desmophen® VP.PU 22HS51 were added to 1.0 g of CNT3 (Baytubes® C150P) in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 80 kJ. The dispersion 3A obtained in this way was used immediately for the further reaction with an isocyanate.

### Example 8

### (Dispersion, 3 Wt. % CNT3, Dispersion 3B)

97 g of Desmophen® VP.PU 22HS51 were added to 3.0 g of CNT3 (Baytubes® C150P) in a 150-ml beaker. This mixture was sheared for 10 minutes at 24,000 rpm with a rotor-stator system (T 18 basic ULTRA-TURRAX®, IKA Werke GmbH & Co. KG, Staufen, Germany) whilst cooling with iced water and then treated with ultrasound (Probe Sonicator HD 3200, BANDELIN electronic GmbH & Co. KG, Berlin, Germany), again whilst cooling with iced water, up to a total energy input of 240 kJ. The dispersion 3B obtained in this way was used immediately for the further reaction with an isocyanate.

### Example 9

### Production of the PU Elastomers

The polyols or the polyol dispersions containing carbon nanotubes (1A, 1B, 2A, 2B, 3A or 3B) were introduced into a 1-litre surface-ground pot and degassed briefly both before and after addition of the catalyst. The isocyanate was stirred briefly at room temperature, the reaction mixture was poured into a hinged metal form and then subjected to an annealing cycle.

Details of the compositions PU1 to PU8 are given in the table below.

| | PU1 | PU2 | PU3 | PU4 | PU5 | PU6 | PU7 | PU8 |
|---|---|---|---|---|---|---|---|---|
| Desmophen ® VP.PU | 99.26 | 96.30 | 99.26 | 96.30 | 99.26 | 96.30 | 99.26 | 96.30 |
| 22HS51 | | | | | | | | |
| CNT1 | | | 1.00 | 2.98 | | | | |
| CNT2 | | | | | 1.00 | 2.98 | | |
| CNT3 | | | | | | | 1.00 | 2.98 |
| DPG | 0.14 | 0.45 | 0.14 | 0.45 | 0.14 | 0.45 | 0.14 | 0.45 |
| DBTL | 0.02 | 0.02 | 0.02 | 0.02 | 0.02 | 0.02 | 0.02 | 0.02 |
| Dabco 33-LV | 0.50 | 0.50 | 0.50 | 0.50 | 0.50 | 0.50 | 0.50 | 0.50 |
| Niax A1 | 0.10 | 0.10 | 0.10 | 0.10 | 0.10 | 0.10 | 0.10 | 0.10 |
| Desmodur ® CD-S | 30.73 | 30.55 | 30.84 | 30.86 | 30.77 | 30.66 | 30.73 | 30.55 |
| Index | 105 | 105 | 105 | 105 | 105 | 105 | 105 | 105 |

The mechanical properties of the elastomers PU1 to PU8 that were obtained are set out in the two tables below.

| | PU1 | PU3 | PU5 | PU7 |
|---|---|---|---|---|
| Nanotube type | — | CNT1 | CNT2 | CNT3 |
| c(Nanotubes) [wt. %] | — | 0.76 | 0.76 | 0.76 |
| Hardness [Shore A] | 55 | 57 | 56 | 55 |
| Tensile strength [MPa] | 2.19 ± 0.22 | 2.52 ± 0.18 | 2.70 ± 0.14 | 2.18 ± 0.32 |
| E modulus [N/mm²] | 1.86 ± 0.09 | 2.49 ± 0.20 | 2.48 ± 0.13 | 1.80 ± 0.17 |
| | PU2 | PU4 | PU6 | PU8 |
| Nanotube type | — | CNT1 | CNT2 | CNT3 |
| c(Nanotubes) [wt. %] | — | 2.27 | 2.27 | 2.27 |
| Hardness [Shore A] | 55 | 63 | 63 | 63 |
| Tensile strength [MPa] | 2.35 ± 0.25 | 2.40 ± 0.17 | 2.66 ± 0.32 | 2.50 ± 0.11 |
| E modulus [N/mm²] | 1.95 ± 0.17 | 3.54 ± 0.15 | 3.94 ± 0.16 | 2.95 ± 0.21 |

To facilitate comparison, the table below shows the percentage changes in the characteristic material values for the elastomers filled with carbon nanotubes as compared with the corresponding unfilled elastomers PU1 and PU2.

| | PU3 | PU4 | PU5 | PU6 | PU7 | PU8 |
|---|---|---|---|---|---|---|
| Δ Hardness [%] | +3.6 | +14.5 | +1.8 | +14.5 | 0 | +14.5 |
| Δ Tensile strength [%] | +15.1 | +2.1 | +23.3 | +13.2 | −0.5 | +6.4 |
| Δ E modulus [%] | +33.9 | +81.5 | +33.3 | +102.1 | −3.2 | +51.3 |

The reinforcing influence of the carbon nanotubes functionalised according to the invention on the polyurethane elastomers is clearly discernible, and the influence is apparent even at relatively low concentrations. In comparison with the unfilled elastomer PU1, the elastomer PU3 containing carbon nanotubes functionalised according to the invention with amine catalysis had a 3.6% greater Shore A hardness, a 15.1% higher tensile strength and a 33.9% higher elasticity modulus. The elastomer PU7 containing entirely non-functionalised carbon nanotubes of the Baytubes® C150P type showed no increase in Shore hardness, a marginal reduction in tensile strength and a reduction in the elasticity modulus as compared with the unfilled elastomer PU1. The reinforcing effect of the carbon nanotubes functionalised according to the invention was even clearer with high filler concentrations. Thus the elastomer PU4 had an 81.5% higher elasticity modulus than the unfilled elastomer PU2. The same filler content with non-functionalised Baytubes C150P, on the other hand, led to only a 51.3% higher elasticity modulus. The reinforcing effect of the carbon nanotubes functionalised according to the invention without amine catalysis in elastomers PU5 and PU6, which had a 33.3% higher and a 102.1% higher elasticity modulus respectively, is also very clear.

The measured viscosities and electrical conductivities of the dispersions that were produced are set out in the table below.

| Dispersion: | * | 1A | 1B | 2A | 2B | 3A | 3B |
|---|---|---|---|---|---|---|---|
| Carbon nanotube type | — | CNT1 | CNT1 | CNT2 | CNT2 | CNT3 | CNT3 |
| Carbon nanotube content | 0 | 1 | 3 | 1 | 3 | 1 | 3 |
| [wt. %] | | | | | | | |
| Electrical conductivity [S/cm] | ** | 3.5E−06 | 3.4E−06 | 7.0E−05 | 6.0E−04 | 3.5E−03 | 6.3E−03 |
| Viscosity [mPa s]*** | 140 | 301 | 1390 | 309 | 1880 | 714 | 2820 |

* Desmophen ® VP.PU 22HS51 without carbon nanotubes;
** below the detection limit;
*** at a shear rate of 100 1/s and 25° C.

The conductivity values listed above clearly show that even functionalisation does not lead to an absolute loss of CNT-typical electrical conductivity but simply reduces it. In the case of dispersions 1A and 1B the conductivity is reduced as compared with the corresponding dispersions 3A and 3B with non-functionalised carbon nanotubes. Furthermore, the filler-induced viscosity rise in dispersions 1A and 1B is only half as great as in 3A and 3B. The same is true of dispersions 2A and 2B in comparison with 3A and 3B. In the case of dispersions 2A and 2B, however, the measurable electrical conductivities are higher than those of 1A and 1B. The difference between these dispersions lies in the way the alkoxylation according to the invention was performed. The type CNT2 carbon nanotubes were alkoxylated in the absence of the amine catalyst. Dispersions 2A and 2B are therefore characterised by a lower viscosity rise in comparison to the non-functionalised carbon nanotubes (3A and 3B) with only a slightly lower electrical conductivity. Thus dispersion types 2A and 2B above all exhibit the ideal preconditions for positively influencing both the mechanical and the electronic properties of polyurethanes produced from them.

1-15. (canceled)

16. Process for producing carbon nanotubes containing hydroxyalkyl ester groups comprising the following steps:
(a) provision of carbon nanotubes having covalently bonded carboxyl groups at their surface; and
(b) reaction of the carbon nanotubes from step (a) with one or more epoxides 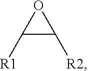
wherein R1 and R2 independently of each other are hydrogen, an alkyl radical or aryl radical and the epoxides are in gaseous form during the reaction.

17. Process according to claim 16, wherein the reaction of the carbon nanotubes in step (b) is performed in the presence of a tertiary amine as catalyst.

18. Process according to claim 16, wherein the reaction of the carbon nanotubes in step (b) is performed in the presence of a tertiary amine that is in gaseous form at the reaction temperature as catalyst.

19. Process according to claim 16, wherein R1 and R2 independently of each other denote hydrogen, methyl, ethyl, n-propyl, isopropyl, n-butyl, sec-butyl, isobutyl, tert-butyl or phenyl or together denote —(CH2)4—.

20. Process according to claim 16, wherein the epoxides used in step (b) are ethylene oxide, propylene oxide, 1,2-butylene oxide or 2,3-butylene oxide.

21. Process according to claim 16, wherein the carbon nanotubes in step (a) are selected from the group encompassing single-walled carbon nanotubes, multi-walled carbon nanotubes, multi-walled carbon nanotubes of the cylinder type, scroll type, multi-scroll type and/or with an onion-like structure.

22. Process according to claim 16, wherein the reaction in step (b) is performed at a temperature of ≧50° C. to ≦200° C.

23. Process according to claim 16, wherein the reaction in step (b) is performed in the presence of an inert gas.

24. Dispersion containing the carbon nanotubes containing hydroxyalkyl ester groups produced by the process according to claim 16 in polyols.

25. Dispersion containing the carbon nanotubes containing hydroxyalkyl ester groups produced by the process according to claim 16 in polyisocyanates and/or NCO-terminated prepolymers.

26. Process for producing the dispersion according to claim 25, wherein the carbon nanotubes are dispersed in the polyisocyanates and/or in the prepolymers, this dispersion is then heated, optionally using a catalyst, to temperatures of between 60° and 150° C., wherein at least some of the terminal free hydroxy groups of the carbon nanotubes react with the polyisocyanate/prepolymer, an additional dispersion is optionally performed, and then it is cooled.

27. Materials containing the carbon nanotubes containing hydroxyalkyl ester groups produced by the process according to claim 16 in a material matrix.

28. Materials according to claim 27, wherein the material matrix is an inorganic polymer, a ceramic material, a polymeric inorganic oxide, a metal or an organic polymer.

29. Materials according to claim 27, wherein the carbon nanotubes are present in a reaction mixture of polyols and polyisocyanates as a matrix.

30. Process for producing a material according to claim 29, comprising the following steps:
(a) provision of a first dispersion of functionalised carbon nanotubes according to claim 1 (i) in a polyol or (ii) in a polyisocyanate,
(b) reaction of this first dispersion (i) with a polyisocyanate or (ii) with a polyol and/or a second dispersion of carbon nanotubes functionalised according to the invention (i) in a polyisocyanate or (ii) in a polyol, optionally in the presence of catalysts, chain extenders, crosslinking agents, auxiliary substances and/or solid or liquid additives commonly used in polyurethane chemistry.
